(19) United States
(12) Patent Application Publication
(10) Pub. No.: US 2009/0118671 A1
(43) Pub. Date: May 7, 2009

(54) Retractable needle device

(75) Inventors: Stephen Francis Goodall, Queensland (AU); Robert Warring Geddes, Queensland (AU); John Milton Moylan, Queensland (AU); Geoffrey Daniel Daly, Queensland (AU)

Correspondence Address: FITCH EVEN TABIN AND FLANNERY, 120 SOUTH LASALLE STREET, SUITE 1600, CHICAGO, IL, 60603-3406, US

(73) Assignee: Analytica Limited, Eight Mile Plains, Queensland (AU)

(21) Appl. No.: 11/911,858

(22) PCT Filed: May 9, 2006
(86) PCT No.: PCT/AU06/00603

§ 371 Date: May 12, 2008

(30) Foreign Application Priority Data
May 9, 2005 (AU) 2005902329

(51) Int. Cl. A61M 5/32

(52) U.S. Cl. 604/138; 604/195

(57) ABSTRACT

A retractable needle device to be attached to the forward end of a syringe. The device has a forward end with a needle assembly that is urged into the body of the device so that when the syringe and device are to be discarded, the needle is located internally of the body of the device to inhibit needles stick injuries.

### TECHNICAL FIELD

The present invention relates to retractable needle devices.

### BACKGROUND OF THE INVENTION

The present invention relates to syringes that when packaged contain a medication to be injected. The present invention also relates to syringes having a retractable needle, such as the syringes described in U.S. Pat. Nos. 5,000,736, 5,125,898, 6,015,438 and 6,179,812 as well as International Patent Publications WO91/10461, WO01/17594, WO00/18454, WO92/18186 and WO98/48869.

The abovementioned syringes have a number of disadvantages including not providing a needle retraction mechanism for a syringe (prefilled) that is delivered and packaged containing a medication to be dispensed.

### OBJECT OF THE INVENTION

It is the object of the present invention to overcome or substantially ameliorate the above disadvantage.

### SUMMARY OF THE INVENTION

There is disclosed herein a retractable needle device including:

a hollow elongated body having a forward end and a rear end, and a bore surrounding a chamber;

a needle assembly releasably mounted at the forward end, the assembly including a needle mounting and a needle, the assembly being configured so as to be receivable within said chamber so as to be contained within said body, said needle being in liquid communication with said chamber;

a resilient member operatively associated with said mounting to urge said assembly into said chamber;

a release member in said chamber movable from a first position to a second position, the second position being spaced from the first position toward said forward end, said release member being operatively associated with said mounting to release said mounting for movement relative to said body when said release member moves to said second position; and wherein said release member is positioned relative to said rear end, when in said first position, to be engaged by a syringe delivering a liquid to said needle via said chamber, the syringe causing movement of said release member to said second position upon a desired quantity of liquid passing from the syringe and being delivered via said needle.

Preferably, said rear end has a socket to engage a forward portion of the syringe, with said release member projecting into said socket rearwardly with respect to said body.

Preferably, said device includes a catch releasably attaching the mounting to the body, with said release member engaging said catch in said second position to release the mounting.

Preferably, said catch includes a resilient clip on said mounting, the clip being movable from an expanded configuration to a retracted configuration by engagement with said release member when said release member moves to said second position, said resilient clip when its in its expanded configuration retains said mounting at said forward end while in said contracted configuration releases said mounting.

Preferably, said clip is a split ring.

Preferably, said release member includes a base extending longitudinally of said chamber and having a rear part engaged by the syringe, and a clip engaging part at a forward part of the base, said clip engaging part engaging said clip to cause operation thereof to release said mounting, said clip engaging part being releasably attached to said forward part and released therefrom subsequent to said clip releasing said mounting so that said clip engaging part moves with said mounting.

Preferably, said clip engaging part includes a plurality of fingers that engage said clip to cause contraction of the clip to release the mounting.

Preferably, said clip engaging part is secured to said forward part by a further clip.

Preferably, said further clip contracts from an expanded configuration to a retracted configuration to release the clip engaging part with respect to said base.

Preferably, said release member projects rearwardly beyond said socket so as to project therefrom to be engaged by a piston of the syringe so that the piston moves the release member to the second positions.

Preferably, said resilient member is compressed between said mounting and body to urge said mounting with said needle into said chamber.

Preferably, said resilient member is a spring.

Preferably, the resilient clip on said mounting is of a frusto-conical configuration providing a head, retaining said mounting at said forward end, and a tapering portion tapering from said head towards said rear end, said tapering portion being engaged by said release member to cause contraction of said head to release said mounting.

Preferably, said release member includes a frusto-conical socket that engages said frusto-conical portion to cause contraction of said head.

Preferably, said head and frusto-conical portion are slotted to provide for contraction of said head.

Preferably, said mounting has a rear portion engaged with a forward portion of said release member to maintain the needle assembly at said forward end, the forward portion of said release member being operable to release the rear portion of said mounting to thereby release said mounting, when said release member is moved to the second position.

Preferably, the release member is resiliently deformed to release the mounting.

Preferably, the mounting is resiliently deformed to release the mounting.

Preferably, the rear portion of said mounting is an annular flange, and the forward portion of said release member is an annular lip, with the lip and flange engaged to sealingly close said space when said release member is in the first position.

Preferably, said release member surrounds a space, with said needle assembly received within said space when released with respect to said body.

Preferably, said space is sealingly closed when said release member is at said first position.

There is further disclosed herein, in combination a syringe and the above device, wherein said syringe has a hollow body containing a piston, the syringe body having a forward end engaged with said rear end, and wherein when the syringe piston is adjacent the syringe forward end the syringe piston is engaged with the release member to cause movement of the release member to said second position.

### BRIEF DESCRIPTION OF THE DRAWINGS

A preferred form of the present invention will now be described by way of example with reference to the accompanying drawings wherein:

FIG. 1 is a schematic side elevation of a syringe and a syringe device;

FIG. 2 is a schematic sectioned side elevation of the retractable needle and device of FIG. 1;

FIGS. 3 to 6 are schematic sectioned side elevations of various operating phases of the syringe and device of FIGS. 1 and 2;

FIG. 7 is a schematic sectioned side elevation of the forward portion of the syringe device of FIG. 1;

FIG. 8 is a schematic sectioned side elevation of a modification of the forward portion of FIG. 7;

FIG. 9 is a schematic sectioned side elevation of a modification of the device of FIG. 1;

FIG. 10 is a schematic sectioned side elevation of a further modification of the device of FIG. 1; and

FIG. 11 is a schematic side elevation of a modification of the device of FIG. 1;

FIG. 12 is a schematic sectioned side elevation of the device of FIG. 11; and

FIG. 13 is a schematic enlarged sectioned side elevation of a forward portion of the device of FIG. 11.

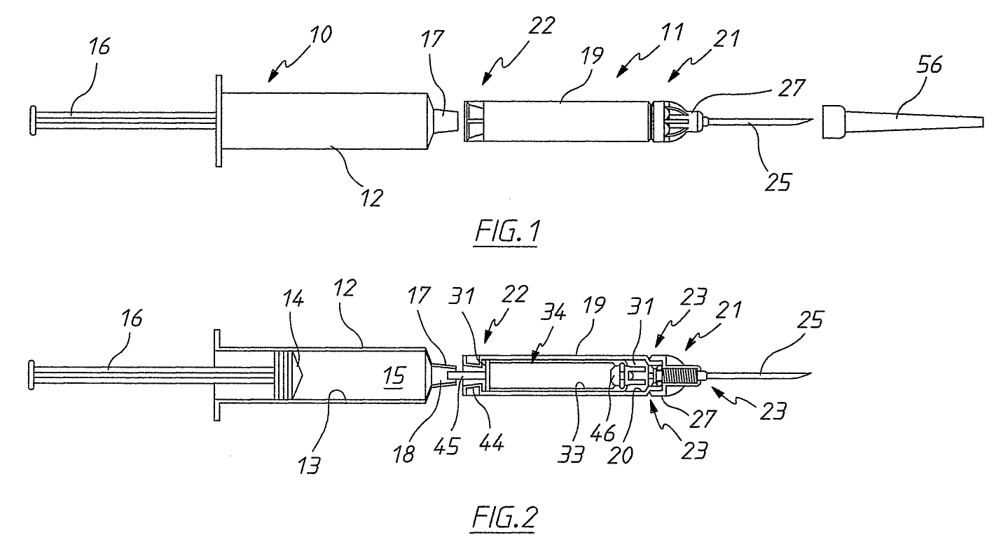

### DETAILED DESCRIPTION OF THE PREFERRED EMBODIMENT

In the accompanying drawings there is schematically depicted a syringe 10 and a retractable needle device 11. The syringe 10 includes a hollow body 12 of generally cylindrical configuration having an internal bore 13. The bore 13 slidably receives a piston 14 that sealingly engages the bore 13 so as to generally enclose a chamber 15 having a liquid medication to be delivered. Extending rearwardly from the piston 14 is a piston rod 16. The forward end of the body 11 tapers to a frusto-conical end 17 surrounding a passage 18. Typically the frusto-conical end 17 would engage a needle mounting and needle if the syringe 10 was to be used without the device 11. In such an arrangement an operator would cause longitudinal movement of the piston 14 by pushing the piston rod 16 toward the end 17. As the volume of the chamber 15 is reduced, liquid in the chamber 15 is forced outwardly through the passage 18.

Operatively associated with the syringe 10 is the syringe device 11. The syringe device 11 includes an elongated hollow body 19 again of generally cylindrical configuration and having a longitudinally extending bore 20. The body 19 has a forward end 21 and a rear end 22, with the forward end 21 releasably containing a needle assembly 23. The needle assembly 23 includes a needle mounting 24 to which there is attached a needle 25.

The body 19 includes at its forward end 21 a cap 27 that is elongated and houses a spring 26. The spring 26 (resilient member) is compressed between the forward portion of the cap 27 and the needle mounting 24 to urge the needle mounting 24 in a direction of the arrow 28. Extending between the mounting 24 and an internal surface 29 of the body 19 is a seal 30 that sealingly closes the forward end 21.

The body 19 encloses a chamber 31 within which the needle assembly 23 is to be received so as to be contained within the body 19.

The spring 26 urges the needle assembly 23 in the direction of the arrow 28 so that the needle 25 does not project forward beyond the forward extremity of the cap 27 when the assembly 23 is released.

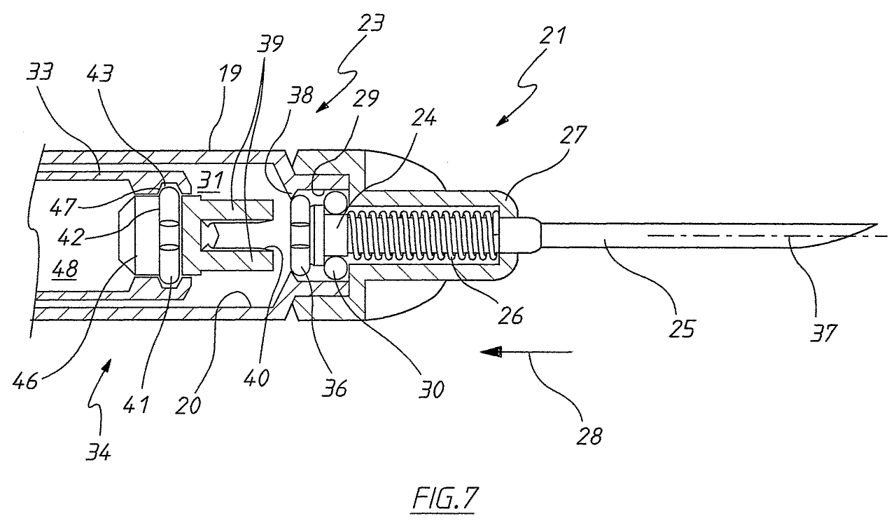

The mounting 24 is retained in the position shown in FIG. 7 by means of a clip 36. The clip 36 of this embodiment is a resilient clip consisting of a split ring that is radially contracted relative to the longitudinal axis 37 from an expanded configuration to a retracted configuration, with the clip 36 in the retracted configuration releasing the mounting 24. In this respect the body 19 has a plurality of radially inwardly projecting ridges 38 that engage the clip 36 when the clip 36 is in the expanded configuration to thereby retain the mounting 24 in the position shown in FIG. 7. However when the clip 36 is radially contracted it is released from the projections 38 to thereby release the mounting 24. The clip 36 is caused to contract by engagement with a release member 34.

The release member 34 includes a clip engaging part in the form of a body 46 with a plurality of forwardly extending fingers 39 that pass between the projections 38 and engage the clip 36 to cause contraction thereof. Each of the fingers 39 has an inclined (ramp) surface 40 that engages the clip 36 to cause contraction thereof.

The release member 34 includes a base 33 having a forward part engaging the body 46 by means of a resilient clip 41. In this embodiment the clip 41 is a split ring. The clip 41 is retained in a groove 42 formed in the body 46 and is radially compressible to release the body 42 from the base 33. More particularly the clip 41 is received in a recess 43 of the base 33. The base 33 is a hollow body that has an internal space 48 that receives at least the mounting 24 and a significant portion of the needle 25, together with the body 46. The body 46 generally closes the space 48 so that the liquid being injected cannot enter the space 48 while the mounting is attached to the body 19.

The rear end 22 of the device 11 is provided with a socket 44 that expands radially outward in a rearward direction and through which a stem 45 of a rear part of the base 33 projects.

In operation of the above described syringe 10 and device 11, a user engages the syringe 10 with the device 11 by inserting the end 17 in the socket 44. The stem 45 projects into the body 12. The piston rod 16 is moved to reduce the volume of the chamber 15 so that the liquid contained in the chamber 15 is caused to enter the chamber 31 and pass between the body 19 and base 33. Air is then expelled from the syringe device 11 through the needle 25. Further movement of the piston rod 16 and therefore piston 14 to reduce the volume of the chamber 15, causes the liquid in the chamber 15 to pass through the chamber 31 and exit via the needle 25.

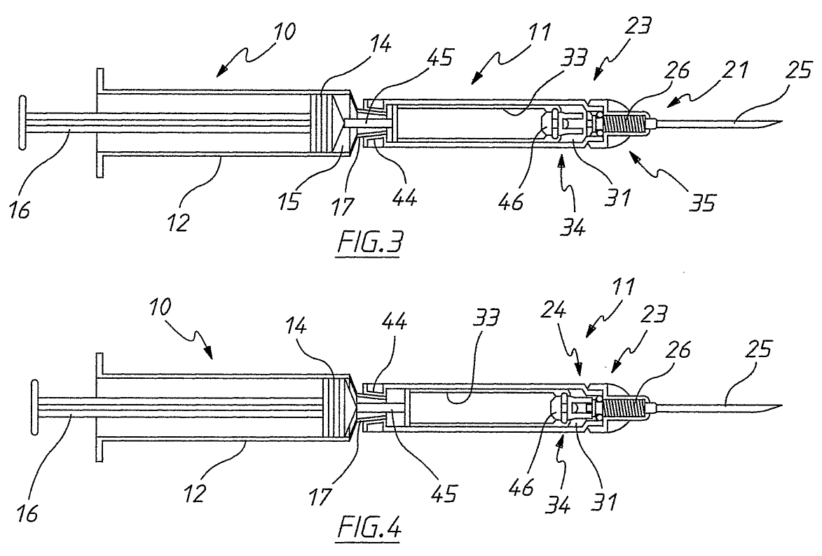

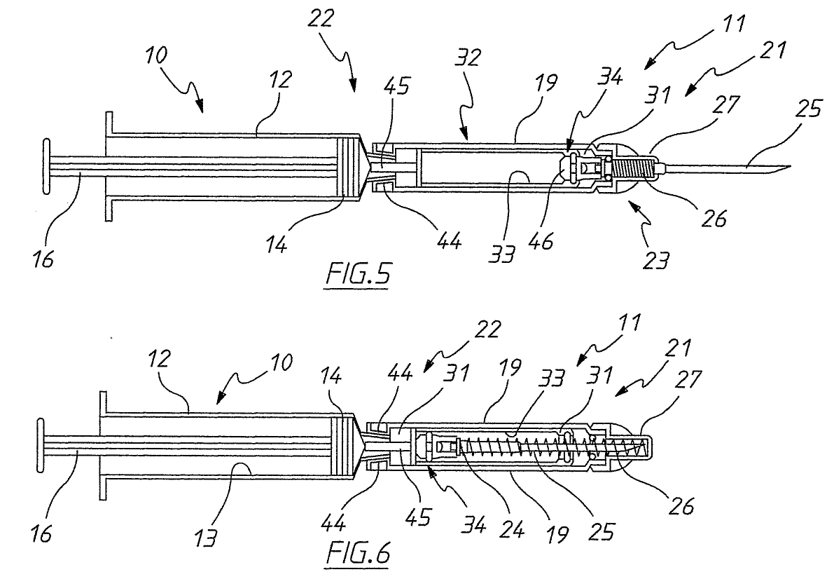

Upon the piston 14 approaching the full extent of its travel, the piston 14 engages the stem 45 and causes movement of the release member 34 from its rear position (first position) to its forward position (second position) as shown in FIGS. 3 and 4. When the release member 34 is moved to its forward position, the fingers 39 engage the clip 36 and cause it to contract thereby releasing the mounting 24. The spring 26 then moves the needle assembly 35 so that it is contained with the body 19, that is, with the needle 25 not projecting beyond the cap 27. However for the mounting 24 to move into the chamber 31, the body 46 must be released from the base 33. This is caused by the fingers 40 being prevented from further forward movement by engagement with the mounting 24 after having released the clip 36. However upon the user continuing to apply pressure to the piston rod 16, the base 33 is caused to move forwardly so that the annular ramp surface 47 of the base 33 causes radial contraction of the clip 41 to release the body 46 from the base 33. Accordingly, the spring 26 also causes the member 34 to move rearwardly with respect to the chamber 31.

Initially the needle would be covered by a cover 56.

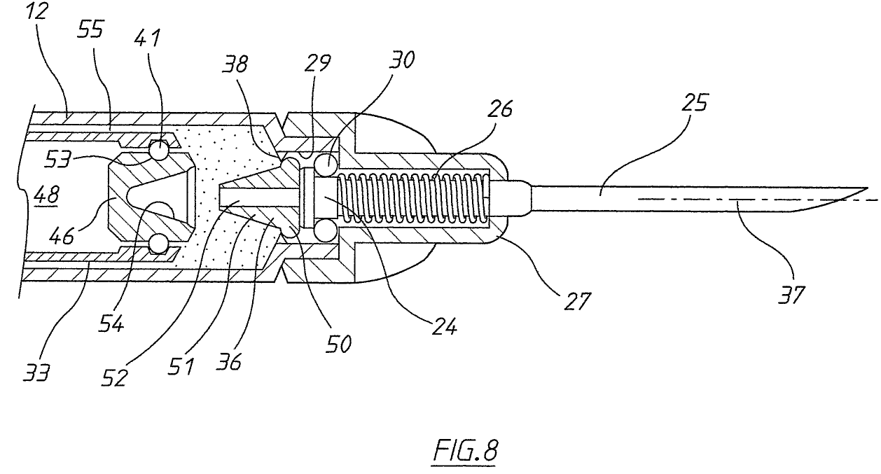

In the embodiment of FIG. 8, the clip 36 is of a moulded plastics material and includes a head 50 that engages the projections 38. Extending rearwardly from the head 50 is a tapering portion 51, with the portion 51 and head being of a generally “frusto-conical” configuration but having a longitudinal slot 52. The body 46 has an annular recess 53 that receives the clip 41. In this embodiment the clip 41 is a resilient “O-ring” that also sealingly closes the space 48 enclosed within the base 33. The “O-ring” is formed of resilient plastics material so as to be compressible. The clip 46 has a frusto-conical internal surface 54 configured to engage the tapering portion 52 to cause radial contraction (relative to the longitudinal axis 37) of the clip 36 and in particular the head 50 so that the head 50 is removed from engagement with the projections 38. This allows movement of the mounting 24 into the base 33 together with at least the majority of the needle 25. As best seen in FIG. 8, there is an annular space 55 between the body 12 and base 33 to provide for the flow of the liquid medication from the syringe 10 to the needle 25. In this respect it should be appreciated the space 48 is sealingly closed by the clip 41. However when the clip 36 is contracted and the body 46 prevented from any further forward movement, the clip 41 is resiliently deformed so that the body 46 is released and enters the space 48 due to further forward movement of the base 33.

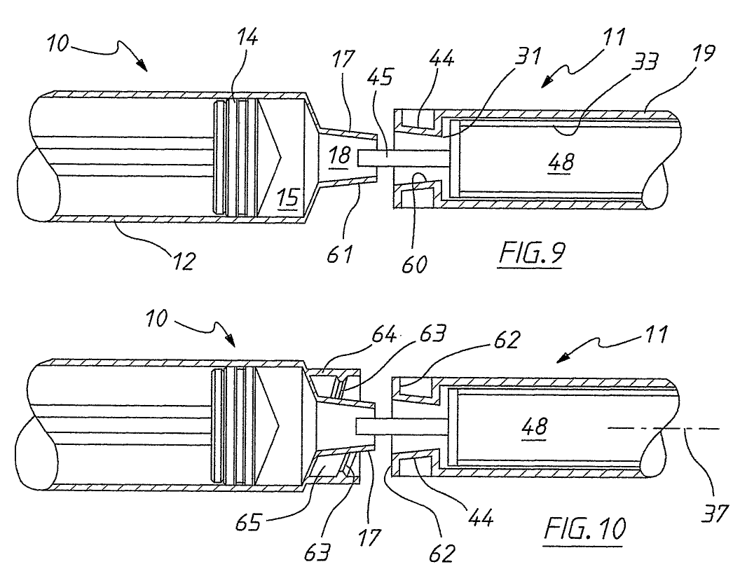

The above described preferred embodiments of the device 11 are intended to be used with luer slip and (FIG. 9) luer lock (FIG. 10) disposable, non-disposable and pre filled syringes.

In FIG. 9 a modification of the device 11 of FIG. 1 is illustrated. In this embodiment the device 11 includes a luer slip arrangement in that it has the socket 44 with a tapering “frusto-conical” surface 60. The surface 60 engages the frusto-conical surface 61 of the end 17. The surfaces 60 and 61 are complimentary and frictionally engaged to securely connect the syringe 10 with the device 11.

In the embodiment of FIG. 10 the device 11 is adapted to engage the syringe 10 by means of a “luer lock”. In this embodiment the socket 44 has an annular flange 62 that engages with the threads 63 that project radially inwardly from a cylindrical flange 64 extending forwardly and coaxially from the body 12. The flange 64 cooperates with the end 17 to provide a cavity 65 that receives the flange 62. When the flange 62 engages the threads 63 the flange 62 is deformed so as to threadably engage the thread 63 and securely connect the device 11 with the syringe 10 by relative rotation of the device 11 with respect to the syringe 10 about the longitudinal axis 37.

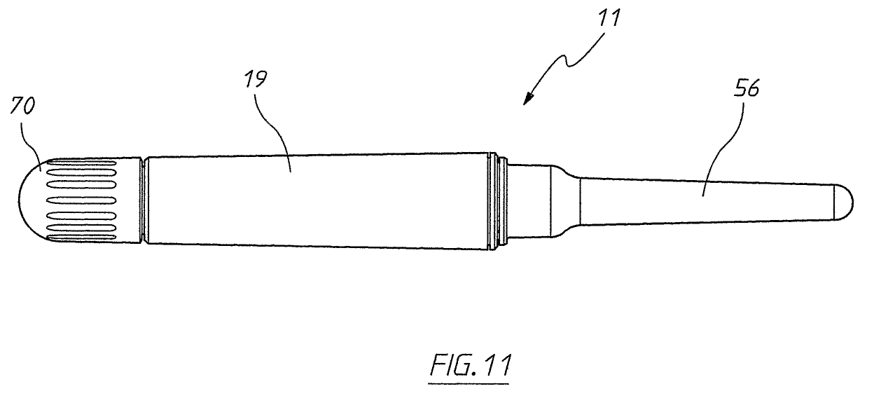

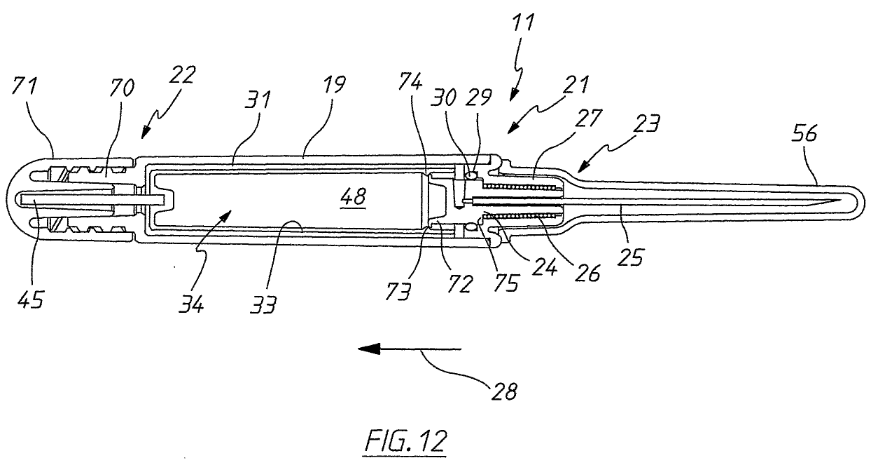

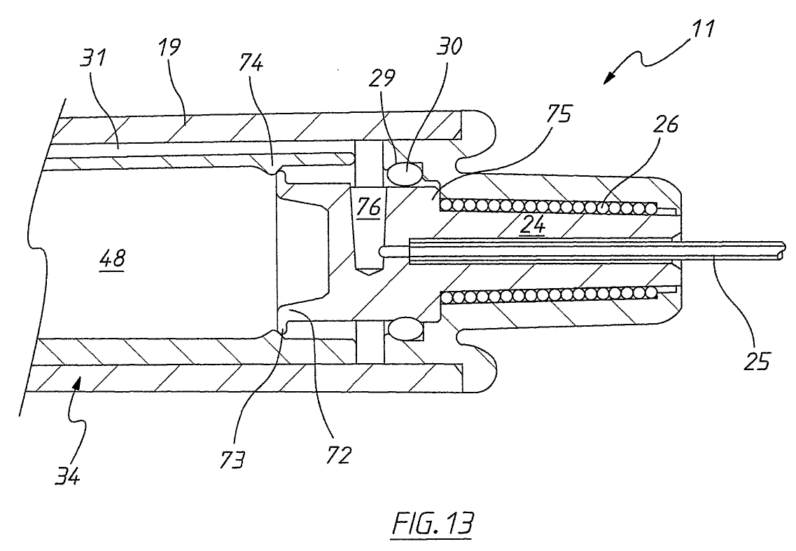

In the embodiment of FIGS. 11 to 13 there is schematically depicted a modification of the device 11. In this embodiment, the body 19 has a rearwardly projecting annular flange 70 to threadably engage an internally threaded similar flange or a modified form of the syringe 10. A cover 71 is provided, that is removed when the syringe 10 is to be engaged.

In this embodiment the mounting 24 has an annular flange 72 with a radially extending portion 73 that engages an annular lip 74 of a forward portion of the base 33. The annular flange 72 is at a rear portion of the mounting 24. The annular flange 72 sealingly engages the lip 74 so as to sealingly close the space 48.

When the stem 45 is engaged by the syringe 10 so as to move the release member 34 towards the mounting 24, the base 3 at adjacent the lip 74 is resiliently deformed radially outward, together with resilient inward deformation of the flange 72, to release the mounting 24 with respect to the base 33 and the body 19. As the mounting 24 moves in the direction of the arrow 28 relative to the body 19, the seal 30 moves past the circular step 75 in the mounting 24 to thereby facilitate movement of the needle assembly 23 into the space 48 and therefore the chamber 31. In this embodiment, the needle assembly 23 is retained in the operative position by being urged in the direction of the arrow 28 so as to abut the lip 74.

The chamber 31 extends to a passage 76 that provides for flow of the liquid being injected to the interior of the needle 25.

1. A retractable needle device including:
a hollow elongated body having a forward end and a rear end, and a bore surrounding a chamber;
a needle assembly releasably mounted at the forward end, the assembly including a needle mounting and a needle, the assembly being configured so as to be receivable within said chamber so as to be contained within said body, said needle being in liquid communication with said chamber;
a resilient member operatively associated with said mounting to urge said assembly into said chamber;
a release member in said chamber movable from a first position to a second position, the second position being spaced from the first position toward said forward end, said release member being operatively associated with said mounting to release said mounting for movement relative to said body when said release member moves to said second position; and wherein said release member is positioned relative to said rear end, when in said first position, to be engaged by a syringe delivering a liquid to said needle via said chamber, the syringe causing movement of said release member to said second position upon a desired quantity of liquid passing from the syringe and being delivered via said needle.

2. The device of claim 1, wherein said rear end has a socket to engage a forward portion of the syringe, with said release member projecting into said socket rearwardly with respect to said body.

3. The device of claim 1, wherein said device includes a catch releasably attaching the mounting to the body, with said release member engaging said catch in said second position to release the mounting.

4. The device of claim 3, wherein said catch includes a resilient clip on said mounting, the clip being movable from an expanded configuration to a retracted configuration by engagement with said release member when said release member moves to said second position, said resilient clip when its in its expanded configuration retains said mounting at said forward end while in said contracted configuration releases said mounting.

5. The clip of claim 4, wherein said clip is a split ring.

6. The device of claim 5, wherein said release member includes a base extending longitudinally of said chamber and having a rear part engaged by the syringe, and a clip engaging part at a forward part of the base, said clip engaging part engaging said clip to cause operation thereof to release said mounting, said clip engaging part being releasably attached to said forward part and released therefrom subsequent to said clip releasing said mounting so that said clip engaging part moves with said mounting.

7. The device of claim 6, wherein said clip engaging part includes a plurality of fingers that engage said clip to cause contraction of the clip to release the mounting.

8. The device of claim 7, wherein said clip engaging part is secured to said forward part by a further clip.

9. The device of claim 8, wherein said further clip contracts from an expanded configuration to a retracted configuration to release the clip engaging part with respect to said base.

10. The device of claim 9, wherein said release member projects rearwardly beyond said socket so as to project therefrom to be engaged by a piston of the syringe so that the piston moves the release member to the second positions.

11. The device of claim 1, wherein said resilient member is compressed between said mounting and body to urge said mounting with said needle into said chamber.

12. The device of claim 1, wherein said resilient member is a spring.

13. The device of claim 4, wherein the resilient clip on said mounting is of a frusto-conical configuration providing a head, retaining said mounting at said forward end, and a tapering portion tapering from said head towards said rear end, said tapering portion being engaged by said release member to cause contraction of said head to release said mounting.

14. The device of claim 13, wherein said release member includes a frusto-conical socket that engages said frusto-conical portion to cause contraction of said head.

15. The device of claim 13, wherein said head and frusto-conical portion are slotted to provide for contraction of said head.

16. The device of claim 1, wherein said mounting has a rear portion engaged with a forward portion of said release member to maintain the needle assembly at said forward end, the forward portion of said release member being operable to release the rear portion of said mounting to thereby release said mounting, when said release member is moved to the second position.

17. The device of claim 16, wherein the release member is resiliently deformed to release the mounting.

18. The device of claim 16, wherein the mounting is resiliently deformed to release the mounting.

19. The device of claim 18, wherein the rear portion of said mounting is an annular flange, and the forward portion of said release member is an annular lip, with the lip and flange engaged to sealingly close said space when said release member is in the first position.

20. The device of claim 1, wherein said release member surrounds a space, with said needle assembly received within said space when released with respect to said body.

21. The device of claim 20, wherein said space is sealingly closed when said release member is at said first position.

22. In combination, a syringe and the device of claim 1, wherein said syringe has a hollow body containing a piston, the syringe body having a forward end engaged with said rear end, and wherein when the syringe piston is adjacent the syringe forward end the syringe piston is engaged with the release member to cause movement of the release member to said second position.
